(19) United States
(12) Patent Application Publication
(10) Pub. No.: US 2015/0030589 A1
(43) Pub. Date: Jan. 29, 2015

(54) ABETA ANTIBODY FORMULATION

(71) Applicant: Hoffmann-La Roche Inc., Little Falls, NJ (US)

(72) Inventors: Pierre Goldbach, Mulhouse (FR); Hanns-Christian Mahler, Basel (CH); Robert Mueller, Basel (CH)

(21) Appl. No.: 14/381,849

(22) PCT Filed: Mar. 5, 2013
(86) PCT No.: PCT/EP2013/054313

§ 371 Date: Aug. 28, 2014

(30) Foreign Application Priority Data
Mar. 8, 2012 (EP) 12158602.8

(51) Int. Cl. A61K 47/34; A61K 47/26; C07K 16/18; A61K 39/395; A61K 47/18

(52) U.S. Cl. CPC A61K 47/34; A61K 39/3955; A61K 47/183; C07K 16/18; A61K 47/26; C07K 2317/565; C07K 2317/51; C07K 2317/515; C07K 2317/41
USPC 424/133.1; 424/139.1

(57) ABSTRACT

The present invention relates to a pharmaceutical formulation comprising about 50 mg/ml-200 mg/ml of an Abeta antibody, about 0.01%-0.1% poloxamer, about 5 mM-50 mM of a buffer, about 100 mM-300 mM of a stabilizer at a pH of about 4.5-7.0.

The present invention relates to a pharmaceutical formulation of an antibody molecule, and/or a mixture of antibody molecules against the amyloid-beta peptide (Abeta).

Antibody molecules, as part of the group of protein pharmaceuticals, are very susceptible to physical and chemical degradation, such as denaturation and aggregation, deamidation, oxidation and hydrolysis. Protein stability is influenced by the characteristics of the protein itself, e.g. the amino acid sequence, and by external influences, such as temperature, solvent pH, excipients, interfaces, or shear rates. So, it is important to define the optimal formulation conditions to protect the protein against degradation reactions during manufacturing, storage and administration. (Manning, M. C., K. Patel, et al. (1989). “Stability of protein pharmaceuticals.” Pharm Res 6(11): 903-18., Zheng, J. Y. and L. J. Janis (2005). “Influence of pH, buffer species, and storage temperature on physicochemical stability of a humanized monoclonal antibody LA298.” Int)_Pharm.). Administration of antibodies via subcutaneous or intramuscular route requires high protein concentration in the final formulation due to the often required high doses and the limited administration volumes. (Shire, S. J., Z. Shahrokh, et al. (2004). “Challenges in the development of high protein concentration formulations.” J Pharm Sci 93(6): 1390-402., Roskos, L. K., C. G. Davis, et al. (2004). “The clinical pharmacology of therapeutic monoclonal antibodies.” Drug Development Research 61(3): 108-120.) The large-scale manufacturing of high protein concentration can be achieved by ultrafiltration processes, drying process, such as lyophilisation or spray-drying, and precipitation processes. (Shire, S.1., Z. Shahrokh, et al. (2004). “Challenges in the development of high protein concentration formulations.” J Pharm Sci 93(6): 1390-402.).

It is an object of the present invention is to provide a highly concentrated, stable formulation of an Abeta antibody or of mixtures of such antibodies, which allows subcutaneous administration of the antibody to a patient.

The formulation of the present invention shows good stability upon storage for 8 months at 2-8° C. and 25° C. without formation of visible particles. Shaking and multiple freezing-thawing steps were applied to the liquid formulation to simulate physical stress conditions that potentially occur during manufacturing or transportation of the drug product.

The pharmaceutical formulation of the present invention comprises a poloxamer as surfactant to reduce aggregation of the antibodies and particle formation. The term “poloxamer” as used herein includes a polyoxyethylene-polyoxypropylene triblock copolymer known asoloxamer 188, sold under the trade name PLURONIC® F68 by BASF (Parsippany, N.J.). Other poloxamers which may be utilized in the formulations of the present invention includeoloxamer 403 (sold as PLURONIC® P123), poloxamer 407 (sold as PLURONIC® P127), oloxamer 402 (sold as PLURONIC® P122), poloxamer 181 (sold as PLURONIC® L61), poloxamer 401 (sold as PLURONIC® L121), poloxamer 185 (sold as PLURONIC® P65), and poloxamer 338 (sold as PLURONIC® F108).

The present invention provides a stable liquid pharmaceutical antibody formulation comprising:
about 50 mg/ml-200 mg/ml of an Abeta antibody,
about 0.01%-0.1% of a poloxamer, preferably poloxamer 188,
about 5 mM-50 mM of a buffer,
about 100 mM-300 mM of a stabilizer,
at a pH of about 4.5-7.0

In a particular embodiment of the present invention, the Abeta antibody concentration is about 100 mg/ml-200 mg/ml, preferably about 150 mg/ml.

In a particular embodiment of the present invention, the poloxamer is present in a concentration of about 0.02%-0.06%, preferably about 0.04%.

In a particular embodiment of the present invention, the buffer is a sodium acetate buffer or a Histidine buffer, preferably a Histidine/Histidine-HCl buffer.

In a particular embodiment of the present invention, the buffer has a concentration of about 10 to 30 mM, preferably about 20 mM.

In a particular embodiment of the present invention, the pH of the formulation is about 5-6, preferably about 5.5.

In a particular embodiment of the present invention, the stabilizer is selected from sugars and amino acids.

In a particular embodiment of the present invention, the stabilizer is selected from trehalose and arginine.

In a particular embodiment of the present invention, the stabilizer has a concentration of about 100 mM to 300 mM.

In a particular embodiment of the present invention, the stabilizer is threhalose and has a concentration of about 150 mM to 250 mM, preferably about 200 mM.

In a particular embodiment of the present invention, the stabilizer is arginine and has a concentration of about 100 mM to 150 mM, preferably about 135 mM.

In a particular embodiment of the present invention, the Abeta antibody is a monoclonal antibody comprising a heavy chain and a light chain.

In a particular embodiment of the present invention, the heavy chain of the Abeta antibody comprises a VH domain which comprises:
a CDR1 comprising the amino acid sequence of Seq. Id. No. 4,
a CDR2 comprising the amino acid sequence of Seq. Id. No. 5,
a CDR3 sequence comprising the amino acid sequence of Seq. Id. No. 6.

In a particular embodiment of the present invention the light chain of the Abeta antibody comprises a VL domain which comprises:
a CDR1 comprising the amino acid sequence of Seq. Id. No. 7,
a CDR2 comprising the amino acid sequence of Seq. Id. No. 8,
a CDR3 sequence comprising the amino acid sequence of Seq. Id. No. 9.

In a particular embodiment of the present invention, the VH domain of the Abeta antibody comprises the amino acid sequence of Seq. Id. No. 2 and the VL domain of the Abeta antibody comprises the amino acid sequence of Seq. Id. No. 3.

In a particular embodiment of the present invention, the heavy chain of the Abeta antibody comprises the amino acid sequence of Seq. Id. No. 10.

In a particular embodiment of the present invention, the light chain of the Abeta antibody comprises the amino acid sequence of Seq. Id. No. 11.

In a particular embodiment of the present invention, the monoclonal Abeta antibody is a mixture of mono-glycosylated Abeta antibodies and double-glycosylated Abeta antibodies, wherein the mono-glycosylated antibody comprises a glycosylated asparagine (Asn) at position 52 of Seq. Id. No. 2 in the VH domain of one antibody binding site and wherein the double-glycosylated antibody comprises a glycosylated asparagine (Asn) at position 52 of Seq. Id. No. 2 in the VH domain of both antibody binding sites and whereby said mixture comprises less than 5% of an antibody being non-glycosylated at position 52 of Seq. Id. No. 2 in the VH domain.

In a particular embodiment the present invention provides the use of the pharmaceutical formulation of the present invention for the subcutaneous administration of the Abeta antibody.

The terms “Abeta antibody” and “an antibody that binds to Abeta” refer to an antibody that is capable of binding Aβ peptide with sufficient affinity such that the antibody is useful as a diagnostic and/or therapeutic agent in targeting Aβ peptide.

It is of note that Aβ has several naturally occurring forms, whereby the human forms are referred to as the above mentioned Aβ39, Aβ40, Aβ41, Aβ42 and Aβ43. The most prominent form, Aβ42, has the amino acid sequence (starting from the N-terminus):

DAEFRHDSGYEVHHQKLVFFAEDVGSNKGAIIGLMVGGVVIA (Seq. Id. No. 1). In Aβ41, Aβ 40, Aβ 39, the C-terminal amino acids A, IA and VIA are missing, respectively. In the Aβ 43 form an additional threonine residue is comprised at the C-terminus of the above depicted sequence (Seq. Id. No. 1).

The term “mono-glycosylated Abeta antibody” relates to an antibody molecule comprising an N-glycosylation at position 52 of Seq. Id. No. 2 in one (VH)-region of an individual antibody molecule; see also FIG. 1. The term “double-glycosylation Abeta antibody” defines an antibody molecule which is N-glycosylated at position 52 of Seq. Id. No. 2 on both variable regions of the heavy chain” (FIG. 1). Antibody molecules which lack a N-glycosylation on both heavy chain (VH)-domains are named “non-glycosylated antibodies” (FIG. 1). The mono-glycosylated antibody, the double-glycosylated antibody and the non-glycosylated antibody may comprise the identical amino acid sequences or different amino acid sequences. The mono-glycosylated antibody and the double-glycosylated antibody are herein referred to as “glycosylated antibody isoforms”. A purified antibody molecule characterized in that at least one antigen binding site comprises a glycosylation in the variable region of the heavy chain (VH) is a mono-glycosylated antibody which is free of or to a very low extent associated with an isoform selected from a double-glycosylated antibody and a nonglycosylated antibody, i.e. a “purified mono-glycosylated antibody”. A double-glycosylated antibody in context of this invention is free of or to a very low extent associated with an isoform selected from a mono-glycosylated antibody and a non-glycosylated antibody, i.e. a “purified double-glycosylated antibody”.

The term “antibody” encompasses the various forms of antibody structures including but not being limited to whole antibodies and antibody fragments. The antibody according to the invention is preferably a humanized antibody, chimeric antibody, or further genetically engineered antibody as long as the characteristic properties according to the invention are retained.

“Antibody fragments” comprise a portion of a full length antibody, preferably the variable domain thereof, or at least the antigen binding site thereof. Examples of antibody fragments include diabodies, single-chain antibody molecules, and multispecific antibodies formed from antibody fragments. scFv antibodies are, e.g. described in Houston, J. S., Methods in Enzymol. 203 (1991) 46-96). In addition, antibody fragments comprise single chain polypeptides having the characteristics of a V_{H} domain, namely being able to assemble together with a V_{L} domain, or of a V_{L} domain binding to Aβ, namely being able to assemble together with a V_{H} domain to a functional antigen binding site and thereby providing the property.

The terms “monoclonal antibody” or “monoclonal antibody composition” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties according to the invention, especially in regard to C1q binding and/or Fc receptor (FcR) binding. Such chimeric antibodies are also referred to as “class-switched antibodies.”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See e.g. Morrison, S. L., et al., Proc. Natl. Acad. Sci. USA 81 (1984) 6851-6855; U.S. Pat. Nos. 5,202,238 and 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See e.g. Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric antibodies. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to C1q binding and/or FcR binding, e.g. by “class switching” i.e. change or mutation of Fc parts (e.g. from IgG1 to IgG4 and/or IgG1/IgG4 mutation.).

The term “epitope” includes any polypeptide determinant capable of specific binding to an antibody. In certain embodiments, epitope determinant include chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody.

The “variable domain” (variable domain of a light chain (V_{L}), variable domain of a heavy chain (V_{H})) as used herein denotes each of the pair of light and heavy chain domains which are involved directly in binding the antibody to the antigen. The variable light and heavy chain domains have the same general structure and each domain comprises four framework (FR) regions whose sequences are widely conserved, connected by three “hypervariable regions” (or complementary determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies according to the invention and therefore provide a further object of the invention.

The term “antigen-binding portion of an antibody” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, Md. (1991) and/or those residues from a “hypervariable loop”.

The term “stabilizer” denotes a pharmaceutical acceptable excipient, which protects the active pharmaceutical ingredient and/or the formulation from chemical and/or physical degradation during manufacturing, storage and application. Chemical and physical degradation pathways of protein pharmaceuticals are reviewed by Cleland, J. L., M. F. Powell, et al. (1993). “The development of stable protein formulations: a close look at protein aggregation, deamidation, and oxidation.” Crit Rev Ther Drug Carrier Syst 10(4): 307-77, Wang, W. (1999). “Instability, stabilization, and formulation of liquid protein pharmaceuticals.” Int J Pharm 185(2): 129-88., Wang, W. (2000). “Lyophilization and development of solid protein pharmaceuticals.” Int J Pharm 203(1-2): 1-60. and Chi, E. Y.,. S. Krishnan, et al. (2003). “Physical stability of proteins in aqueous solution: mechanism and driving forces in nonnative protein aggregation.” Pharm Res 20(9): 1325-36. Stabilizers include but are not limited to sugars, amino acids, polyols, surfactants, antioxidants, preservatives, cyclodextrines, polyethylenglycols, e.g. PEG 3000, 3350, 4000, 6000, albumin, e.g. human serum albumin (HSA), bovines serum albumin (BSA), salts, e.g. sodium chloride, magnesium chloride, calcium chloride, chelators, e.g. EDTA as hereafter defined. As mentioned hereinabove, stabilizers can be present in the formulation in an amount of about 10 to about 500 mM, preferably in an amount of about 10 to about 300 mM and more preferably in an amount of about 100 mM to about 300 mM.

A “stable liquid pharmaceutical antibody formulation” is a liquid antibody formulation with no significant changes observed at a refrigerated temperature (2-8° C.) for at least 12 months, particularly 2 years, and more particularly 3 years. The criteria for stability are the following: no more than 10%, particularly 5%, of antibody monomer is degraded as measured by size exclusion chromatography (SEC-HPLC). Furthermore, the solution is colorless or clear to slightly opalescent by visual analysis. The protein concentration of the formulation has no more than +/−10% change. No more than 10%, particularly 5% of aggregation is formed. The stability is measured by methods known in the art such UV spectroscopy, size exclusion chromatography (SEC-HPLC), Ion-Exchange Chromatography (IE-HPLC), turbidimetry and visual inspection.

Recombinant Methods and Compositions

Antibodies may be produced using recombinant methods and compositions, e.g., as described in U.S. Pat. No. 4,816,567. In one embodiment, isolated nucleic acid encoding an anti-[[PRO]] antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-[[PRO]] antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-Abeta antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### EXAMPLES

Liquid drug product formulations for subcutaneous administration according to the invention were developed as follows.

### Example 1

### Preparation of Liquid Formulations

The following Abeta liquid formulations were prepared at a protein concentration of 150 mg/ml:

| Code | Buffer | Surfactant | Excipient |
|---|---|---|---|
| F1 | 20 mM Sodium | 0.02% Polysorbate 20 | 200 mM Trehalose |
| F2 | Acetate pH 5.5 | 0.02% Polysorbate 20 | 210 mM Sorbitol |
| F3 | | 0.02% Polysorbate 20 | 135 mM Arginine |
| F4 | | 0.02% Polysorbate 80 | 200 mM Trehalose |
| F5 | | 0.02% Polysorbate 80 | 210 mM Sorbitol |
| F6 | | 0.02% Polysorbate 80 | 135 mM Arginine |
| F7 | | 0.04% Poloxamer 188 | 200 mM Trehalose |
| F8 | | 0.04% Poloxamer 188 | 135 mM Arginine |
| F9 | 20 mM Histidine/ | 0.02% Polysorbate 20 | 200 mM Trehalose |
| F10 | Histidine-HCl | 0.02% Polysorbate 20 | 210 mM Sorbitol |
| F11 | pH 5.5 | 0.02% Polysorbate 20 | 135 mM Arginine |
| F12 | | 0.02% Polysorbate 80 | 200 mM Trehalose |
| F13 | | 0.02% Polysorbate 80 | 210 mM Sorbitol |
| F14 | | 0.02% Polysorbate 80 | 135 mM Arginine |
| F15 | | 0.04% Poloxamer 188 | 200 mM Trehalose |
| F16 | | 0.04% Poloxamer 188 | 135 mM Arginine |

Abeta antibody prepared and obtained as described in WO2007/068429 was provided at a concentration of approx. 50-60 mg/mL in a 10 mM histidine buffer at a pH of approx. 5.5. The Abeta antibody used in the examples comprises the CDRs, VH domain, VL domain, heavy chain and light chain specified in the Sequence Listing of the present application (Seq. Id. No. 2-11).

For the preparation of the liquid formulations Abeta was buffer-exchanged against a diafiltration buffer containing the anticipated buffer composition and concentrated by ultrafiltration to an antibody concentration of approx. 200 mg/mL. After completion of the ultrafiltration operation, the excipients (e.g. trehalose) were added as stock solutions to the antibody solution. The surfactant was then added as a 50 to 125-fold stock solution. Finally the protein concentration was adjusted with a buffer to the final Abeta concentration of approx. 150 mg/mL.

All formulations were sterile-filtered through 0.22 μm low protein binding filters and aseptically filled into sterile 6 mL glass vials closed with ETFE (Copolymer of ethylene and tetrafluoroethylene)-coated rubber stoppers and alucrimp caps. The fill volume was approx. 2.4 mL. These formulations were stored at different climate conditions (5° C., 25° C. and 40° C.) for different intervals of time and stressed by shaking (1 week at a shaking frequency of 200 min-1 at 5° C. and 25° C.) and freeze-thaw stress methods (five cycles at −80° C./+5° C.). The samples were analyzed before and after applying the stress tests as well as after storage by the following analytical methods:
UV spectroscopy
Size Exclusion Chromatography (SEC)
Ion exchange chromatography (IEC)
Clarity and opalescence of the solution
Visual inspection

UV spectroscopy, used for determination of protein content, was performed on a Perkin Elmer λ35 UV spectrophotometer in a wavelength range from 240 nm to 400 nm. Neat protein samples were diluted to approx. 0.5 mg/mL with the corresponding formulation buffer. The protein concentration was calculated according to equation 1.

$$Protein content = \frac{A ( 280 ) - A ( 320 ) \times dil . factor}{ɛ 〈 {cm}^{2} \text{/} mg 〉 \times d 〈 cm 〉} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient E.

Size Exclusion Chromatography (SEC) was used to detect soluble high molecular weight species (aggregates) and low molecular weight hydrolysis products (LMW) in the formulations. The method was performed on a Waters Alliance 2695 HPLC instrument with a Waters W2487 Dual Absorbance Detector and equipped with a TosoHaas TSK-Gel G3000SWXL column. Intact monomer, aggregates and hydrolysis products were separated by an isocratic elution profile, using 0.2M K2HPO4/0.25M KCL, pH 7.0 as mobile phase, and were detected at a wavelength of 280 nm.

Ion Exchange Chromatography (IEC) was performed to detect chemical degradation products altering the net charge of Abeta in the formulations. The method used a Waters Alliance 2695 HPLC instrument with a Waters W2487 Dual Absorbance Detector and equipped (detection wavelength 280 nm) and a Mono S TM 5/50GL column (Amersham Biosciences). 50 mM malonic acid/malonate pH 5.3 and 1M Na-acetate in Mobile Phase A pH 5.3 used as mobile phases A and B, respectively, with a flow rate of 1.0 mL/min.

Gradient Program:

| min | Mobile Phase A | Mobile Phase B |
|---|---|---|
| 0 | 100 | 0 |
| 1 | 100 | 0 |
| 20 | 48 | 52 |
| 22 | 48 | 52 |
| 24 | 0 | 100 |
| 25 | 0 | 100 |
| 26 | 100 | 0 |
| 30 | 100 | 0 |

Clarity and the degree of opalescence were measured as Formazine Turbidity Units (FTU) by the method of nephelometry. The neat sample was transferred into a 11 mm diameter clearglass tube and placed into a HACH 2100AN turbidimeter.

Samples were inspected for the presence of visible particles by using a Seidenader V90-T visual inspection instrument.

Compositions and Stability Data of Liquid Abeta Drug Product Formulations According to this Invention
F1 is a liquid formulation with the composition 150 mg/mL Abeta, 20 mM sodium acetate, 200 mM trehalose, 0.02% polysorbate 20, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 151.8 | 2.3 | 97.0 | 0.7 | 23.7 | 23.7 | 3.3 | Practically free from particles |
| Shaking 5° C. | 1 week | — | 2.3 | 96.7 | 1.0 | — | — | 3.5 | Practically free from particles |
| Shaking 25° C. | 1 week | 150.0 | 2.3 | 96.9 | 0.9 | — | — | 3.3 | Practically free from particles |
| Freeze/thaw | 5 cycles | — | 2.4 | 96.8 | 0.8 | — | — | 3.8 | Practically free from particles |
| 5° C. | 8 months | — | 2.8 | 97.1 | 0.1 | 23.2 | 23.2 | 6.5 | With many particles |
| 25° C. | 8 months | — | 3.5 | 94.5 | 2.0 | 15.8 | 15.8 | 6.5 | With many particles |
| 40° C. | 8 months | 152.0 | 9.5 | 79.6 | 11.0 | 3.4 | 3.4 | 7.8 | With many particles |

F2 is a liquid formulation with the composition 150 mg/mL Abeta, 20 mM sodium acetate, 210 mM sorbitol, 0.02% polysorbate 20, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 153.2 | 2.2 | 97.2 | 0.7 | 45.3 | 23.7 | 3.6 | Practically free from particles |
| Shaking 5° C. | 1 week | — | 2.3 | 96.9 | 0.8 | — | — | 3.6 | Practically free from particles |
| Shaking 25° C. | 1 week | 152.1 | 2.3 | 97.1 | 0.8 | — | — | 3.9 | Practically free from particles |
| Freeze/thaw | 5 cycles | — | 2.2 | 96.9 | 0.8 | — | — | 4.2 | Practically free from particles |
| 5° C. | 8 months | — | 2.7 | 97.2 | 0.1 | 47.7 | 23.1 | 7.8 | With many particles |
| 25° C. | 8 months | — | 3.6 | 94.4 | 2.1 | 61.0 | 15.6 | 7.0 | With many particles |
| 40° C. | 8 months | 151.8 | 10.3 | 78.6 | 11.1 | 67.9 | 3.5 | 7.5 | With many particles |

F3 is a liquid formulation with the composition 150 mg/mL Abeta, 20 mM sodium acetate, 135 mM Arginine, 0.02% polysorbate 20, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 154.5 | 1.8 | 97.5 | 0.7 | 45.8 | 23.8 | 11.4 | Practically free from particles |
| Shaking 5° C. | 1 week | — | 1.9 | 97.3 | 0.9 | — | — | 11.9 | Practically free from particles |
| Shaking 25° C. | 1 week | 153.1 | 1.9 | 97.4 | 0.8 | — | — | 11.3 | Practically free from particles |
| Freeze/thaw | 5 cycles | — | 1.8 | 97.3 | 0.8 | — | — | 10.9 | Practically free from particles |
| 5° C. | 8 months | — | 2.3 | 97.6 | 0.1 | 23.4 | 13.3 | 13.3 | With many particles |
| 25° C. | 8 months | — | 2.7 | 95.1 | 2.2 | 17.6 | 13.4 | 13.4 | With many particles |
| 40° C. | 8 months | 152.1 | 8.6 | 78.5 | 12.9 | 4.8 | 12.1 | 12.1 | With many particles |

F4 is a liquid formulation with the composition 150 mg/mL Abeta, 20 mM sodium acetate, 200 mM trehalose, 0.02% polysorbate 80, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 152.3 | 2.2 | 97.1 | 0.7 | 45.3 | 23.8 | 3.8 | Practically free from particles |
| Shaking 5° C. | 1 week | — | 2.2 | 97.0 | 0.8 | — | — | 3.4 | Practically free from particles |
| Shaking 25° C. | 1 week | 151.4 | 2.2 | 97.1 | 0.7 | — | — | 3.8 | Practically free from particles |
| Freeze/thaw | 5 cycles | — | 2.4 | 96.8 | 0.8 | — | — | 3.5 | Practically free from particles |
| 5° C. | 8 months | — | 2.8 | 97.1 | 0.1 | 47.7 | 23.2 | 3.6 | Practically free from particles |
| 25° C. | 8 months | — | 3.5 | 94.5 | 2.0 | 60.7 | 15.7 | 5.2 | With many particles |
| 40° C. | 8 months | 149.3 | 9.8 | 79.0 | 11.2 | 68.0 | 3.5 | 7.2 | With many particles |

F5 is a liquid formulation with the composition 150 mg/mL Abeta, 20 mM sodium acetate, 210 mM sorbitol, 0.02% polysorbate 80, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 153.4 | 2.0 | 97.4 | 0.6 | 45.9 | 23.8 | 4.2 | Practically free from particles |
| Shaking 5° C. | 1 week | — | 2.1 | 97.0 | 0.9 | — | — | 4.2 | Practically free from particles |
| Shaking 25° C. | 1 week | 153.1 | 2.1 | 97.1 | 0.8 | — | — | 4.0 | Practically free from particles |
| Freeze/thaw | 5 cycles | — | 2.3 | 97.0 | 0.8 | — | — | 4.0 | Practically free from particles |
| 5° C. | 8 months | — | 2.8 | 97.2 | 0.1 | 47.7 | 23.2 | 4.2 | Practically free from particles |
| 25° C. | 8 months | — | 3.5 | 94.5 | 2.0 | 60.9 | 15.6 | 5.6 | With many particles |
| 40° C. | 8 months | 151.1 | 10.2 | 78.7 | 11.1 | 67.9 | 3.5 | 9.4 | With many particles |

F6 is a liquid formulation with the composition 150 mg/mL Abeta, 20 mM sodium acetate, 135 mM Arginine, 0.02% polysorbate 80, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 155.2 | 1.7 | 97.7 | 0.6 | 46.0 | 23.8 | 11.1 | Practically free from particles |
| Shaking 5° C. | 1 week | — | 1.8 | 97.5 | 0.8 | — | — | 10.9 | Practically free from particles |
| Shaking 25° C. | 1 week | 152.0 | 1.7 | 97.6 | 0.7 | — | — | 11.1 | Practically free from particles |
| Freeze/thaw | 5 cycles | — | 1.9 | 97.3 | 0.8 | — | — | 11.0 | Practically free from particles |
| 5° C. | 8 months | — | 2.3 | 97.7 | 0.1 | 46.7 | 23.3 | 10.6 | With many particles |
| 25° C. | 8 months | — | 2.7 | 95.0 | 2.2 | 57.2 | 17.6 | 11.1 | Practically free from particles |
| 40° C. | 8 months | 152.9 | 9.1 | 77.9 | 13.1 | 65.6 | 4.9 | 12.0 | With many particles |

F7 is a liquid formulation with the composition 150 mg/mL Abeta, 20 mM sodium acetate, 200 mM trehalose, 0.04% poloxamer 188, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 149.5 | 2.0 | 97.9 | 0.2 | 49.9 | 23.9 | 3.88 | Practically free from particles |
| Shaking 5° C. | 1 week | — | 2.1 | 97.8 | 0.1 | — | — | 3.50 | Practically free from particles |
| Shaking 25° C. | 1 week | 147.75 | 2.2 | 97.6 | 0.2 | — | — | 3.32 | Practically free from particles |
| Freeze/thaw | 5 cycles | — | 2.1 | 97.8 | 0.1 | — | — | 3.43 | Practically free from particles |
| 5° C. | 8 months | — | 2.4 | 97.2 | 0.4 | 53.1 | 23.8 | 13.3 | Practically free from particles |
| 25° C. | 8 months | — | 3.3 | 94.0 | 2.8 | 62.3 | 17.1 | 4.14 | Practically free from particles |
| 40° C. | 8 months | 151.20 | 10.3 | 77.0 | 12.7 | 69.0 | 2.4 | 4.70 | Practically free from particles |

F8 is a liquid formulation with the composition 150 mg/mL Abeta, 20 mM sodium acetate, 135 mM Arginine, 0.04% poloxamer 188, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 148.90 | 1.7 | 98.1 | 0.1 | 50.5 | 23.9 | 12.9 | Practically free from particles |
| Shaking 5° C. | 1 week | — | 1.8 | 98.0 | 0.1 | — | — | 12.6 | Practically free from particles |
| Shaking 25° C. | 1 week | 144.89 | 1.9 | 98.0 | 0.2 | — | — | 12.2 | Practically free from particles |
| Freeze/thaw | 5 cycles | — | 1.8 | 98.0 | 0.1 | — | — | 13.30 | Practically free from particles |
| 5° C. | 8 months | — | 2.0 | 97.6 | 0.4 | 51.6 | 23.9 | 15.1 | Practically free from particles |
| 25° C. | 8 months | — | 2.6 | 94.4 | 3.0 | 59.2 | 19.0 | 13.1 | Practically free from particles |
| 40° C. | 8 months | 150.32 | 9.6 | 75.3 | 15.1 | 66.0 | 3.9 | 17.1 | Practically free from particles |

F9 is a liquid formulation with the composition 150 mg/mL Abeta, 20 mM histidine, 200 mM trehalose, 0.02% polysorbate 20, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 150.7 | 1.7 | 97.7 | 0.7 | 44.9 | 23.7 | 4.4 | Practically free from particles |
| Shaking 5° C. | 1 week | — | 1.7 | 97.6 | 0.7 | — | — | 4.0 | Practically free from particles |
| Shaking 25° C. | 1 week | 149.0 | 1.7 | 97.5 | 0.7 | — | — | 4.7 | Practically free from particles |
| Freeze/thaw | 5 cycles | — | 1.9 | 97.3 | 0.8 | — | — | 4.4 | Practically free from particles |
| 5° C. | 8 months | — | 2.2 | 97.7 | 0.1 | 47.7 | 23.3 | 7.4 | With many particles |
| 25° C. | 8 months | — | 2.9 | 95.0 | 2.1 | 58.3 | 17.9 | 7.7 | With many particles |
| 40° C. | 8 months | 150.2 | 9.0 | 78.3 | 12.8 | 66.7 | 5.1 | 7.4 | With many particles |

F10 is a liquid formulation with the composition 150 mg/mL Abeta, 20 mM histidine, 210 mM sorbitol, 0.02% polysorbate 20, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 152.6 | 1.7 | 97.7 | 0.7 | 46.3 | 23.7 | 4.82 | Practically free from particles |
| Shaking 5° C. | 1 week | — | 1.7 | 97.6 | 0.7 | — | — | 4.80 | Practically free from particles |
| Shaking 25° C. | 1 week | 151.4 | 1.7 | 97.6 | 0.7 | — | — | 4.45 | Practically free from particles |
| Freeze/thaw | 5 cycles | — | 1.9 | 97.4 | 0.8 | — | — | 4.67 | Practically free from particles |
| 5° C. | 8 months | — | 2.2 | 97.7 | 0.1 | 47.5 | 23.3 | 7.38 | With many particles |
| 25° C. | 8 months | — | 2.9 | 95.1 | 2.0 | 58.5 | 17.8 | 7.69 | With many particles |
| 40° C. | 8 months | 152.0 | 8.7 | 78.4 | 12.9 | 66.5 | 5.1 | 7.48 | With many particles |

F11 is a liquid formulation with the composition 150 mg/mL Abeta, 20 mM histidine, 135 mM Arginine, 0.02% polysorbate 20, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 154.1 | 1.5 | 97.9 | 0.6 | 45.2 | 23.8 | 11.5 | Practically free from particles |
| Shaking 5° C. | 1 week | — | 1.6 | 97.7 | 0.7 | — | — | 11.4 | Practically free from particles |
| Shaking 25° C. | 1 week | 152.0 | 1.6 | 97.7 | 0.7 | — | — | 10.8 | Practically free from particles |
| Freeze/thaw | 5 cycles | — | 1.7 | 97.6 | 0.8 | — | — | 11.4 | Practically free from particles |
| 5° C. | 8 months | — | 2.0 | 97.9 | 0.1 | 46.6 | 23.4 | 13.1 | With many particles |
| 25° C. | 8 months | — | 2.4 | 95.4 | 2.2 | 55.8 | 18.7 | 15.0 | With many particles |
| 40° C. | 8 months | 153.5 | 8.0 | 77.7 | 14.3 | 66.5 | 5.1 | 11.8 | With many particles |

F12 is a liquid formulation with the composition 150 mg/mL Abeta, 20 mM histidine, 200 mM trehalose, 0.02% polysorbate 80, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 151.0 | 1.7 | 97.6 | 0.7 | 45.5 | 23.7 | 4.53 | Practically free from particles |
| Shaking 5° C. | 1 week | — | 1.7 | 97.6 | 0.7 | — | — | 4.40 | Practically free from particles |
| Shaking 25° C. | 1 week | 151.3 | 1.7 | 97.5 | 0.8 | — | — | 4.20 | Practically free from particles |
| Freeze/thaw | 5 cycles | — | 2.0 | 97.2 | 0.8 | — | — | 4.41 | Practically free from particles |
| 5° C. | 8 months | — | 2.2 | 97.7 | 0.1 | 47.7 | 23.3 | 4.43 | With many particles |
| 25° C. | 8 months | — | 2.9 | 94.9 | 2.1 | 58.3 | 17.9 | 6.24 | With many particles |
| 40° C. | 8 months | 150.9 | 9.1 | 78.2 | 12.8 | 66.8 | 5.1 | 9.88 | With many particles |

F13 is a liquid formulation with the composition 150 mg/mL Abeta, 20 mM histidine, 210 mM sorbitol, 0.02% polysorbate 80, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 153.2 | 1.7 | 97.6 | 0.7 | 46.1 | 23.7 | 4.68 | Practically free from particles |
| Shaking 5° C. | 1 week | — | 1.7 | 97.6 | 0.7 | — | — | 4.47 | Practically free from particles |
| Shaking 25° C. | 1 week | 152.2 | 1.8 | 97.5 | 0.7 | — | — | 4.73 | Practically free from particles |
| Freeze/thaw | 5 cycles | — | 1.9 | 97.3 | 0.8 | — | — | 4.54 | Practically free from particles |
| 5° C. | 8 months | — | 2.2 | 97.7 | 0.1 | 47.5 | 23.3 | 5.24 | With many particles |
| 25° C. | 8 months | — | 2.9 | 95.0 | 2.1 | 58.5 | 17.8 | 6.33 | With many particles |
| 40° C. | 8 months | 152.1 | 8.8 | 78.2 | 13.0 | 66.6 | 5.2 | 10.8 | With many particles |

F14 is a liquid formulation with the composition 150 mg/mL Abeta, 20 mM histidine, 135 mM Arginine, 0.02% polysorbate 80, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 154.7 | 1.6 | 97.7 | 0.8 | 45.7 | 23.8 | 11.3 | Practically free from particles |
| Shaking 5° C. | 1 week | — | 1.6 | 97.7 | 0.7 | — | — | 10.9 | Practically free from particles |
| Shaking 25° C. | 1 week | 153.0 | 1.6 | 97.6 | 0.8 | — | — | 11.8 | Practically free from particles |
| Freeze/thaw | 5 cycles | — | 1.8 | 97.5 | 0.8 | — | — | 10.9 | Practically free from particles |
| 5° C. | 8 months | — | 2.0 | 97.9 | 0.1 | 46.7 | 23.3 | 11.1 | With many particles |
| 25° C. | 8 months | — | 2.5 | 95.3 | 2.3 | 55.9 | 18.7 | 11.4 | With many particles |
| 40° C. | 8 months | 155.1 | 8.5 | 76.7 | 14.7 | 64.2 | 6.6 | 13.3 | With many particles |

F15 is a liquid formulation with the composition 150 mg/mL Abeta, 20 mM histidine, 200 mM trehalose, 0.04% poloxamer 188, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 151.1 | 1.8 | 98.1 | 0.2 | 50.5 | 23.9 | 4.94 | Practically free from particles |
| Shaking 5° C. | 1 week | — | 1.9 | 98.0 | 0.1 | — | — | 4.42 | Practically free from particles |
| Shaking 25° C. | 1 week | 150.7 | 2.0 | 97.8 | 0.2 | — | — | 4.06 | Practically free from particles |
| Freeze/thaw | 5 cycles | — | 1.9 | 98.0 | 0.1 | — | — | 4.25 | Practically free from particles |
| 5° C. | 8 months | — | 2.1 | 97.5 | 0.4 | 51.9 | 23.9 | n.d. | Practically free from particles |
| 25° C. | 8 months | — | 2.9 | 94.2 | 2.9 | 59.6 | 19.6 | 5.40 | Practically free from particles |
| 40° C. | 8 months | 152.3 | 9.7 | 74.4 | 15.9 | 66.5 | 5.0 | 6.36 | Practically free from particles |

F16 is a liquid formulation with the composition 150 mg/mL Abeta, 20 mM histidine, 135 mM Arginine, 0.04% poloxamer 188, at pH 5.5

| | | Protein | Size Exclusion-HPLC | | | Ion Exchange-HPLC | | | |
|---|---|---|---|---|---|---|---|---|---|
| Storage | Storage | conc. | HMW | Monomer | LMW | Peak 1 | Peak 2 | Turbidity | |
| condition | Time | (mg/mL) | (%) | (%) | (%) | (%) | (%) | (FTU) | Visible particles |
| — | Initial | 151.8 | 1.6 | 98.2 | 0.2 | 50.4 | 23.9 | 13.0 | Practically free from particles |
| Shaking 5° C. | 1 week | — | 1.7 | 98.1 | 0.1 | — | — | 12.5 | Practically free from particles |
| Shaking 25° C. | 1 week | 147.9 | 1.8 | 98.1 | 0.2 | — | — | 12.7 | Practically free from particles |
| Freeze/thaw | 5 cycles | — | 1.7 | 98.1 | 0.1 | — | — | 12.8 | Practically free from particles |
| 5° C. | 8 months | — | 1.9 | 97.7 | 0.4 | 51.8 | 23.9 | 16.8 | Practically free from particles |
| 25° C. | 8 months | — | 2.3 | 94.6 | 3.1 | 57.5 | 20.6 | 12.9 | Practically free from particles |
| 40° C. | 8 months | 152.2 | 8.9 | 72.5 | 18.6 | 62.6 | 7.3 | 14.2 | Practically free from particles |

The stability data presented above show that all of the polysorbate 20 and polysorbate 80 containing formulations are developing visible particles after 8 months storage at 5° C., 25° C. or 40° C. On the other hand, the poloxamer containing formulations are practically free from visible particles after storage for 8 months at 5° C., 25° C. and 40° C. Therefore poloxamer is able to prevent the formation of visible particles in Abeta antibody formulations.

### Amino Acid Sequences Disclosed in the Application

| Amino acid sequence | Seq. Id. No. |
|---|---|
| Abeta peptide Aβ | 1 |
| VH domain of Abeta antibody | 2 |
| VL domain of Abeta antibody | 3 |
| CDR1 of VH domain of Abeta antibody | 4 |
| CDR2 of VH domain of Abeta antibody | 5 |
| CDR3 of VH domain of Abeta antibody | 6 |
| CDR1 of VL domain of Abeta antibody | 7 |
| CDR2 of VL domain of Abeta antibody | 8 |
| CDR3 of VL domain of Abeta antibody | 9 |
| Heavy chain Abeta antibody | 10 |
| Light chain Abeta antibody | 11 |

1. A stable liquid pharmaceutical antibody formulation comprising:
about 50 mg/ml-200 mg/ml of an Abeta antibody,
about 0.01%-0.1% of a poloxamer, preferably poloxamer 188,
about 5 mM-50 mM of a buffer,
about 100 mM-300 mM of a stabilizer,
wherein the formulation has a pH of about 4.5-7.0

2. The pharmaceutical formulation according to claim 1, wherein the Abeta antibody concentration is about 100 mg/ml-200 mg/ml.

3-18. (canceled)

19. The formulation according to claim 2, wherein the Abeta antibody concentration is about 150 mg/ml.

20. The pharmaceutical formulation according to claim 1, wherein the poloxamer is present in a concentration of about 0.02%-0.06%.

21. The pharmaceutical formulation according to claim 1, wherein the poloxamer is present in a concentration of about 0.02%-0.06%.

22. The formulation according to claim 19, wherein the poloxamer is about 0.04%.

23. The pharmaceutical formulation according to claim 1, wherein the buffer is a sodium acetate buffer or a Histidine buffer.

24. The pharmaceutical formulation according to claim 23, wherein the buffer is a Histidine buffer.

25. The pharmaceutical formulation according to claim 2, wherein the buffer is a sodium acetate buffer or a Histidine buffer.

26. The pharmaceutical formulation according to claim 3, wherein the buffer is a sodium acetate buffer or a Histidine buffer.

27. The pharmaceutical formulation according to claim 19, wherein the buffer is a sodium acetate buffer or a Histidine buffer.

28. The pharmaceutical formulation according to claim 20, wherein the buffer is a sodium acetate buffer or a Histidine buffer.

29. The pharmaceutical formulation according to claim 21, wherein the buffer is a sodium acetate buffer or a Histidine buffer.

30. The pharmaceutical formulation according to claim 22, wherein the buffer is a sodium acetate buffer or a Histidine buffer.

31. The pharmaceutical formulation according to claim 1, wherein the buffer has a concentration of about 10 to 30 mM.

32. The pharmaceutical formulation according to claim 2, wherein the buffer has a concentration of about 10 to 30 mM.

33. The pharmaceutical formulation according to claim 23, wherein the buffer has a concentration of about 10 to 30 mM.

34. The pharmaceutical formulation according to claim 24, wherein the buffer has a concentration of about 10 to 30 mM.

35. The pharmaceutical formulation according to claim 25, wherein the buffer has a concentration of about 10 to 30 mM.

36. The pharmaceutical formulation according to claim 26, wherein the buffer has a concentration of about 10 to 30 mM.

37. The pharmaceutical formulation according to claim 27, wherein the buffer has a concentration of about 10 to 30 mM.

38. The pharmaceutical formulation according to claim 22, wherein the buffer has a concentration of about 10 to 30 mM.

39. The pharmaceutical formulation according to claim 1, wherein the pH of the formulation is about 5-6.

40. The pharmaceutical formulation according to claim 2, wherein the pH of the formulation is about 5-6.

41. The pharmaceutical formulation according to claim 19, wherein the pH of the formulation is about 5-6.

42. The pharmaceutical formulation according to claim 20, wherein the pH of the formulation is about 5-6.

43. The pharmaceutical formulation according to claim 19, wherein the pH of the formulation is about 5-6.

44. The pharmaceutical formulation according to claim 20, wherein the pH of the formulation is about 5-6.

45. The pharmaceutical formulation according to claim 21, wherein the pH of the formulation is about 5-6.

46. The pharmaceutical formulation according to claim 22, wherein the pH of the formulation is about 5-6.

47. The pharmaceutical formulation according to claim 23, wherein the pH of the formulation is about 5-6.

48. The pharmaceutical formulation according to claim 24, wherein the pH of the formulation is about 5-6.

49. The pharmaceutical formulation according to claim 25, wherein the pH of the formulation is about 5-6.

50. The pharmaceutical formulation according to claim 26, wherein the pH of the formulation is about 5-6.

51. The pharmaceutical formulation according to claim 27, wherein the pH of the formulation is about 5-6.

52. The pharmaceutical formulation according to claim 28, wherein the pH of the formulation is about 5-6.

53. The pharmaceutical formulation according to claim 29, wherein the pH of the formulation is about 5-6.

54. The pharmaceutical formulation according to claim 1, wherein the stabilizer is selected from sugars and amino acids.

55. The pharmaceutical formulation according to claim 2, wherein the stabilizer is selected from trehalose and arginine.

56. The pharmaceutical formulation according to claim 19, wherein the stabilizer is arginine and has a concentration of about 100 mM to 150 mM.

57. The pharmaceutical formulation according to claim 20, wherein the stabilizer is arginine and has a concentration of about 100 mM to 150 mM.

58. The pharmaceutical formulation according to claim 21, wherein the stabilizer is arginine and has a concentration of about 100 mM to 150 mM.

59. The pharmaceutical formulation according to claim 22, wherein the stabilizer is arginine and has a concentration of about 100 mM to 150 mM.

60. The pharmaceutical formulation according to claim 1, wherein the Abeta antibody is a monoclonal antibody comprising a heavy chain and a light chain.

61. The pharmaceutical formulation according to claim 60, wherein the heavy chain of the Abeta antibody comprises a VH domain which comprises:
a CDR1 comprising the amino acid sequence of Seq. Id. No. 4,
a CDR2 comprising the amino acid sequence of Seq. Id. No. 5, and
a CDR3 sequence comprising the amino acid sequence of Seq. Id. No. 6.

62. The pharmaceutical formulation according to claim 60, wherein the light chain of the Abeta antibody comprises a VL domain which comprises:
a CDR1 comprising the amino acid sequence of Seq. Id. No. 7,
a CDR2 comprising the amino acid sequence of Seq. Id. No. 8,
a CDR3 sequence comprising the amino acid sequence of Seq. Id. No. 9.

63. The pharmaceutical formulation according to claim 60, wherein the light chain of the Abeta antibody comprises a VL domain which comprises:
a CDR1 comprising the amino acid sequence of Seq. Id. No. 7,
a CDR2 comprising the amino acid sequence of Seq. Id. No. 8,
a CDR3 sequence comprising the amino acid sequence of Seq. Id. No. 9.

64. The pharmaceutical formulation according to claim 60, wherein the heavy chain of the Abeta antibody comprises a VH domain which comprises:
a CDR1 comprising the amino acid sequence of Seq. Id. No. 4,
a CDR2 comprising the amino acid sequence of Seq. Id. No. 5, and
a CDR3 sequence comprising the amino acid sequence of Seq. Id. No. 6.

65. The pharmaceutical formulation according to claim 60, wherein the VH domain of the Abeta antibody comprises the amino acid sequence of Seq. Id. No. 2 and the VL domain of the Abeta antibody comprises the amino acid sequence of Seq. Id. No. 3.

66. The pharmaceutical formulation according to claim 60, wherein the heavy chain of the Abeta antibody comprises the amino acid sequence of Seq. Id. No. 10.

67. The pharmaceutical formulation according to claim 60, wherein the heavy chain of the Abeta antibody comprises the amino acid sequence of Seq. Id. No. 10.

68. The pharmaceutical formulation according to claim 60, wherein light chain of the Abeta antibody comprises the amino acid sequence of Seq. Id. No. 11.

69. The pharmaceutical formulation according to claim 60, wherein the monoclonal Abeta antibody comprises: a mixture of mono-glycosylated Abeta antibodies and double-glycosylated Abeta antibodies; and wherein the mono-glycosylated antibody comprises a glycosylated asparagine (Asn) at position 52 of Seq. Id. No. 2 in the VH domain of one antibody binding site and wherein the double-glycosylated antibody comprises a glycosylated asparagine (Asn) at position 52 of Seq. Id. No. 2 in the VH domain of both antibody binding sites and whereby said mixture comprises less than 5% of an antibody being non-glycosylated at position 52 of Seq. Id. No. 2 in the VH domain.

70. The pharmaceutical formulation according to claim 60, wherein the monoclonal Abeta antibody comprises: a mixture of mono-glycosylated Abeta antibodies and double-glycosylated Abeta antibodies; and wherein the mono-glycosylated antibody comprises a glycosylated asparagine (Asn) at position 52 of Seq. Id. No. 2 in the VH domain of one antibody binding site and wherein the double-glycosylated antibody comprises a glycosylated asparagine (Asn) at position 52 of Seq. Id. No. 2 in the VH domain of both antibody binding sites and whereby said mixture comprises less than 5% of an antibody being non-glycosylated at position 52 of Seq. Id. No. 2 in the VH domain.

71. The pharmaceutical formulation according to claim 60, wherein the monoclonal Abeta antibody comprises: a mixture of mono-glycosylated Abeta antibodies and double-glycosylated Abeta antibodies; and wherein the mono-glycosylated antibody comprises a glycosylated asparagine (Asn) at position 52 of Seq. Id. No. 2 in the VH domain of one antibody binding site and wherein the double-glycosylated antibody comprises a glycosylated asparagine (Asn) at position 52 of Seq. Id. No. 2 in the VH domain of both antibody binding sites and whereby said mixture comprises less than 5% of an antibody being non-glycosylated at position 52 of Seq. Id. No. 2 in the VH domain.

72. The pharmaceutical formulation according to claim 60, wherein the monoclonal Abeta antibody comprises: a mixture of mono-glycosylated Abeta antibodies and double-glycosylated Abeta antibodies; and wherein the mono-glycosylated antibody comprises a glycosylated asparagine (Asn) at position 52 of Seq. Id. No. 2 in the VH domain of one antibody binding site and wherein the double-glycosylated antibody comprises a glycosylated asparagine (Asn) at position 52 of Seq. Id. No. 2 in the VH domain of both antibody binding sites and whereby said mixture comprises less than 5% of an antibody being non-glycosylated at position 52 of Seq. Id. No. 2 in the VH domain.

73. The pharmaceutical formulation according to claim 60, wherein the monoclonal Abeta antibody comprises: a mixture of mono-glycosylated Abeta antibodies and double-glycosylated Abeta antibodies; and wherein the mono-glycosylated antibody comprises a glycosylated asparagine (Asn) at position 52 of Seq. Id. No. 2 in the VH domain of one antibody binding site and wherein the double-glycosylated antibody comprises a glycosylated asparagine (Asn) at position 52 of Seq. Id. No. 2 in the VH domain of both antibody binding sites and whereby said mixture comprises less than 5% of an antibody being non-glycosylated at position 52 of Seq. Id. No. 2 in the VH domain.

74. The pharmaceutical formulation according to claim 60, wherein the monoclonal Abeta antibody comprises: a mixture of mono-glycosylated Abeta antibodies and double-glycosylated Abeta antibodies; and wherein the mono-glycosylated antibody comprises a glycosylated asparagine (Asn) at position 52 of Seq. Id. No. 2 in the VH domain of one antibody binding site and wherein the double-glycosylated antibody comprises a glycosylated asparagine (Asn) at position 52 of Seq. Id. No. 2 in the VH domain of both antibody binding sites and whereby said mixture comprises less than 5% of an antibody being non-glycosylated at position 52 of Seq. Id. No. 2 in the VH domain.


SEQUENCE LISTING (11 sequences)

SEQ ID NO: 1 - protein, 41 aa, Artificial Sequence
  Abeta 42 peptide
DAEFRHDSGYEVHHQKLVFFAEDVGSNKGAIIGLMVGGWIA

SEQ ID NO: 2 - protein, 126 aa, Artificial Sequence [sequence omitted]

SEQ ID NO: 3 - protein, 110 aa, Artificial Sequence
  VL domain Abeta Antibody
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRT

SEQ ID NO: 4 - protein, 10 aa, Artificial Sequence
  VH CDR1 Abeta Antibody
GFTFSSYAMS

SEQ ID NO: 5 - protein, 17 aa, Artificial Sequence
  VH CDR2 Abeta Antibody
AINASGTRTYYADSVKG

SEQ ID NO: 6 - protein, 17 aa, Artificial Sequence
  VH CDR3 Abeta Antibody
GKGNTHKPYGYVRYFDV

SEQ ID NO: 7 - protein, 12 aa, Artificial Sequence
  VL CDR1 Abeta Antibody
RASQSVSSSYLA

SEQ ID NO: 8 - protein, 7 aa, Artificial Sequence
  VL CDR2 Abeta Antibody
GASSRAT

SEQ ID NO: 9 - protein, 8 aa, Artificial Sequence
  VL CDR3 Abeta Antibody
LQIYNMPI

SEQ ID NO: 10 - protein, 459 aa, Artificial Sequence
  Heavy chain Abeta Antibody
QVELVESGGGLVQPGGSLRLSCAASGFTFSSYAMSWVRQAPGKGLEWVSAINASNASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 215 aa, Artificial Sequence
  Light chain Abeta Antibody
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC